(19) United States
(12) Patent Application Publication
(10) Pub. No.: US 2007/0185163 A1
(43) Pub. Date: Aug. 9, 2007

(54) Imidazol derivatives of piperidine as histamine antagonists

(75) Inventors: Fraser Hunt, Leicestershire (GB); Peter Hamley, Frankfurt (DE)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: AstraZeneca AB, Sodertalje (SE)

(21) Appl. No.: 10/565,464

(22) PCT Filed: Jul. 19, 2004
(86) PCT No.: PCT/GB04/03111

§ 371 Date: Jul. 12, 2006

(30) Foreign Application Priority Data
Jul. 21, 2003 (SE) 0302116-9

(51) Int. Cl. A61K 31/445; C07D 401/06

(52) U.S. Cl. 514/326; 546/210

(57) ABSTRACT

Piperidine compounds according to Formula I 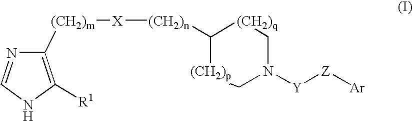
wherein R¹, m, X, n, p, q, Y, Z and Ar are acs defined in the specification, processes and intermediates used in their preparation, pharmaceutical compositions containing them and their use in therapy and in the preparation of medicaments.

### FIELD OF THE INVENTION

The present invention relates to piperidine compounds, processes and intermediates used in their preparation, pharmaceutical compositions containing them and their use in therapy.

### BACKGROUND OF THE INVENTION

Histamine is a biogenic amine that regulates a variety of physiological and pathological processes including inflammation, gastric acid secretion and neurotransmission. Histamine acts via a family of G-protein coupled receptors and 4 members of this family have been identified and cloned: histamine H1 (Yamashita et al, 1991), histamine H2 (Gantz et al, 1991), histamine H3 (Lovenberg et al, 1991) and histamine H4 receptor (Oda et al, 1999). H1 and H2 are the best characterised of these receptors and antagonists of both are used clinically. In general, inflammatory and allergic responses are modified by H1 receptors (Ash and Schild, 1966) while gastric acid secretion is regulated by interaction with H2 receptors (Black et al, 1972). H1 antagonists are therefore used to treat a variety of allergic conditions and H2 antagonists are used to treat gastric ulcers. Histamine appears to regulate neurotransmitter release via H3 receptors (Arrang et al, 1983), but the role of the recently identified histamine H4 receptor is currently unknown. The histamine H4 receptor bears sequence and pharmacological similarity to the H3 receptor, although the tissue distribution profiles of both receptors are different. The H3 receptor is abundant in the brain and neural tissue while the H4 receptor appears to be restricted to peripheral tissues. The H4 receptor has a high distribution in peripheral blood leukocytes, especially eosinophils and neutrophils and H4 mRNA expression has also been demonstrated in other immune and inflammatory cells, including T-cells, dendritic cells, monocytes, macrophages, mast cells and epithelial cells. In addition, there is some evidence that receptor expression may be modulated by cytokine activation (Morse et al, 2001). The H4 receptor may therefore have a role in immune and/or inflammatory modulation.

Histamine H1 receptor antagonists are successfully used in the treatment of allergic rhinitis but provide incomplete blockade of all symptoms resulting in the need for coadministration of other agents to treat nasal congestion, usually sympathomimetic amine decongestants. Combinations of H1 and H2 antagonists also fail to give complete blockade of these effects. Similarly, although histamine contributes to many of the physiological processes that occur in asthma, histamine H1 antagonists are not used in asthma because of inconsistent efficacy. Some further anti-inflammatory activity appears to be required to block the effects of histamine in many patho-physiological processes, implying a role for additional pro-inflammatory histamine receptors. The H4 receptor may serve such a role, and agents that interact with H4 receptors either alone, or in combination with other histamine receptors or anti-inflammatory agents, may provide enhanced efficacy in disease.

Antagonists of the histamine H4 receptor may therefore have utility in a variety of diseases or disorders. WO 02/072548 discloses a series of compounds said to be active as mediators of the histamine H4 receptor.

### DESCRIPTION OF THE INVENTION

In one aspect the present invention provides a compound of formula (I) and pharmaceutically acceptable salts and solvates thereof for use in the manufacture of a medicament and for use for the treatment of diseases mediated by histamine H3 and H4:

Compounds of the invention are those according to formula (I) 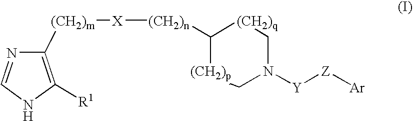
in which:

Ar is an aryl group, a 5-7 membered heteraromatic ring containing 14 heteroatoms selected from nitrogen, oxygen or sulphur, or a bicyclic or tricyclic heteraromatic ring containing 1-4 heteroatoms selected from nitrogen, oxygen or sulphur, each of which can be optionally substituted by 1-3 groups selected from C₁₋₆ alkyl, C₁₋₆ alkylthio, C₁₋₆ alkoxy, halogen, cyano, CF₃, OCF₃, C₃₋₄ cyclolalkyl, C₂₋₆ alkenyl, C₂₋₄ alkynyl, C₂₋₄ alkenyloxy, hydroxyl, nitro, tosyl, thienyl, benzyl, phenyl, nitrophenyl,

R¹ is hydrogen or C₁₋₄ alkyl;

X is O, NR², CH₂ or SOₓ

R² is C₁₋₆ alkyl;

x is 0, 1 or 2;

Y is CH₂, C═O, SO₂, or (C═O)NH;

Z is (CR³R⁴)ᵣ or Y and Z together form a CH═CH group;

m and n are independently 0, 1, 2 or 3;

p and q are independently 0, 1 or 2;

r is 0, 1, 2, 3, or 4 and

R³ and R⁴ are independently hydrogen or C₁₋₆alkyl.

Certain compounds of formula (I) are capable of existing in stereoisomeric forms.

It will be understood that the invention encompasses all geometric and optical isomers of the compounds of formula (I) and mixtures thereof including racemates. Tautomers and mixtures thereof also form an aspect of the present invention.

The term aryl includes phenyl and naphthyl. The term alkyl, whether alone or as part of another group, includes straight chain and branched chain alkyl groups. Examples of 5- to 7-membered heteroaromatic ring containing 1 to 4 heteroatoms include thienyl, furanyl, pyrrolyl, imidazolyl, pyridyl, pyrazinyl, pyrimidyl, pyridazinyl, triazinyl, oxazolyl, thiazolyl, isoxazolyl, pyrazolyl, oxadiazolyl, thiadiazolyl, triazolyl and tetrazolyl. Examples of suitable bicyclic rings include indole, benzothiphene, quinoline, benzodioxan, and naphthyl. Examples of suitable tricyclic rings include dibenzofuran and thiene[2,3-b]benzothiophene. For any of these mono-bicyclic or tricyclic rings, substituents can be present in any suitable ring position including suitable substituents on nitrogen atoms.

In compounds of the invention for use in the preparation of medicaments or for use in the treatment of diseases mediated by histamine H3 and H4:

Preferably Ar¹ is phenyl, furyl or thienyl optionally substituted as defined above. More preferably Ar¹ is phenyl optionally substituted as defined above. Preferred substituents include halogen such as iodo, chloro and flouro, cyclohexyl, methyl, ethyl, propyl, t-butyl, ethynyl, propenyloxy, hydroxyl, methoxy, nitro, tosyl, trifluoromethyl, thienyl, benzyl, cyano, phenylethynyl, nitrophenyl, methylthio, propoxy, butoxy, 2-propenyl, or trifluomethoxy.

Most preferably Ar¹ is phenyl substituted by bromo, hydroxyl or 2,4-difluoro.

Preferably R¹ is hydrogen or methyl.

Preferably X is O.

Preferably Y is CH₂ or C═O and Z is CH₂, CHMe, CH₂CHMe or Y and Z form a CH═CH group.

More preferably Y is CH₂ or C═O and Z is CH₂.

Preferably m is 1 and n is 0.

Preferably p and q are both 1.

Preferred compounds of the invention for use in the preparation of a medicament or for the treatment of diseases mediated by histamine H3 and H4 include:
4-(1H-imidazol-4-ylmethoxy)-1-(1-oxo-3-phenylbutyl)-piperidine
4-(1H-Imidazol-4-ylmethoxy)-1-[[4-(trifluoromethyl)phenyl]acetyl]-piperidine
1-[2-(4-Hydroxyphenyl)-1-oxopropyl]-4-[(5-methyl-1H-imidazol-4-yl)methoxy]-piperidine
1-[(4-fluorophenyl)acetyl]-4-(1H-imidazol-4-ylmethoxy)-piperidine
1-[(2-chlorophenyl)acetyl]-4-(1H-imidazol-4-ylmethoxy)-piperidine
1-[(4-chlorophenyl)acetyl]-4-(1H-imidazol-4-ylmethoxy)-piperidine
4-(1H-imidazol-4-ylmethoxy)-1-(phenylacetyl)-piperidine
1-(4-cyclohexylbenzoyl)-4-(1H-imidazol-4-ylmethoxy)-piperidine
1-[(3,4-dichlorophenyl)acetyl]-4-(1H-imidazol-4-ylmethoxy)-piperidine
4-(1H-imidazol-4-ylmethoxy)-1-[(4-methylphenyl)acetyl]-piperidine
1-[(3,4-difluorophenyl)acetyl]-4-(1H-imidazol-4-ylmethoxy)-piperidine
1-[(2,4-difluorophenyl)acetyl]-4-(1H-imidazol-4-ylmethoxy)-piperidine
4-(1H-imidazol-4-ylmethoxy)-1-[(4′-propyl[1,1′-biphenyl]-4-yl)carbonyl]-piperidine
1-[2-(4-hydroxyphenyl)-1-oxopropyl]-4-(1H-imidazol-4-ylmethoxy)-piperidine
1-[(2E)-3-(3,4-dichlorophenyl)-1-oxo-2-propenyl]-4-(1H-imidazol-4-ylmethoxy)-piperidine
1-[3-(2,4-dichlorophenyl)-1-oxopropyl]-4-(1H-imidazol-4-ylmethoxy)-piperidine
1-[(2,4-dichlorophenyl)acetyl]-4-(1H-imidazol-4-ylmethoxy)-piperidine
1-[(2-Bromophenyl)methyl]-4-(1H-imidazol-4-ylmethoxy)-piperidine
1-[(3-Bromo-2-thienyl)methyl]-4-[(5-methyl-1H-imidazol-4-yl)methoxy]-piperidine
1-[(3-bromo-2-thienyl)methyl]-4-(1H-imidazol-4-ylmethoxy)-piperidine
1-[(4-ethynylphenyl)methyl]-4-(1H-imidazol-4-ylmethoxy)-piperidine
4-(1H-imidazol-4-ylmethoxy)-1-[[3-(4-methylphenoxy)phenyl]methyl]-piperidine
4-(1H-imidazol-4-ylmethoxy)-1-[[4-(2-propenyloxy)phenyl]methyl]-piperidine
4-[[4-(1H-imidazol-4-ylmethoxy)-1-piperidinyl]methyl]-phenol
4-(1H-imidazol-4-ylmethoxy)-1-[(2-methoxyphenyl)methyl]-piperidine
4-(1H-imidazol-4-ylmethoxy)-1-[[3-(4-methoxyphenoxy)phenyl]methyl]-piperidine
1-[(2,3-dichlorophenyl)methyl]-4-(1H-imidazol-4-ylmethoxy)-piperidine
1-[(2-chloro-4-fluorophenyl)methyl]-4-(1H-imidazol-4-ylmethoxy)-piperidine
1-(2-dibenzofuranylmethyl)-4-(1H-imidazol-4-ylmethoxy)-piperidine
4-(1H-imidazol-4-ylmethoxy)-1-[[2-(methylthio)phenyl]methyl]-piperidine
4-(1H-imidazol-4-ylmethoxy)-1-(thieno[2,3-b][1]benzothien-2-ylmethyl)-piperidine
1-[(2-chloro-5-nitrophenyl)methyl]-4-(1H-imidazol-4-ylmethoxy)-piperidine
1H-pyrrole, 2-[[4-(1H-imidazol-4-ylmethoxy)-1-piperidinyl]methyl]-1-[(4-methylphenyl)sulfonyl]
2-ethoxy-6-[[4-(1H-imidazol-4-ylmethoxy)-1-piperidinyl]methyl]-phenol
(1,3-benzodioxol-5-ylmethyl)-4-(1H-imidazol-4-ylmethoxy)-piperidine
4-(1H-imidazol-4-ylmethoxy)-1-[[4-(phenylmethoxy)phenyl]methyl]-piperidine
1-[[2-fluoro-4-(trifluoromethyl)phenyl]methyl]-4-(1H-imidazol-4-ylmethoxy)-piperidine
1-[(4-bromophenyl)methyl]-4-(1H-imidazol-4-ylmethoxy)-piperidine
4-(1H-imidazol-4-ylmethoxy)-1-[(4-methylphenyl)methyl]-piperidine
4-(1H-imidazol-4-ylmethoxy)-1-(2-thienylmethyl)-piperidine
1-[(4-chlorophenyl)methyl]-4-(1H-imidazol-4-ylmethoxy)-piperidine
1-[(2-chloro-6-fluorophenyl)methyl]-4-(1H-imidazol-4-ylmethoxy)-piperidine
4-(1H-imidazol-4-ylmethoxy)-1-[(3-methyl-2-thienyl)methyl]-piperidine
4-(1H-imidazol-4-ylmethoxy)-1-(2-naphthalenylmethyl)-piperidine
4-(1H-imidazol-4-ylmethoxy)-1-(1-naphthalenylmethyl)-piperidine
4-(1H-imidazol-4-ylmethoxy)-1-[(2-nitrophenyl)methyl]-piperidine
4-(1H-imidazol-4-ylmethoxy)-1-(3-thienylmethyl)-piperidine
1-([1,1′-biphenyl]-4-ylmethyl)-4-(1H-imidazol-4-ylmethoxy)-piperidine
1-[(2,5-difluorophenyl)methyl]-4-(1H-imidazol-4-ylmethoxy)-piperidine
4-(1H-imidazol-4-ylmethoxy)-1-[(3-phenoxyphenyl)methyl]-piperidine
4-(1H-imidazol-4-ylmethoxy)-1-[(3-methylphenyl)methyl]-piperidine
1-(2-furanylmethyl)-4-(1H-imidazol-4-ylmethoxy)-piperidine
1-[(2,6-dichlorophenyl)methyl]-4-(1H-imidazol-4-ylmethoxy)-piperidine
1-[(4-fluorophenyl)methyl]-4-(1H-imidazol-4-ylmethoxy)-piperidine
1-[(3-fluorophenyl)methyl]-4-(1H-imidazol-4-ylmethoxy)-piperidine
1-(3-furanylmethyl)-4-(1H-imidazol-4-ylmethoxy)-piperidine
1-[(4-ethylphenyl)methyl]-4-(1H-imidazol-4-ylmethoxy)-piperidine
4-(1H-imidazol-4-ylmethoxy)-1-[(2-methylphenyl)methyl]-piperidine
1-[(3-chlorophenyl)methyl]-4-(1H-imidazol-4-ylmethoxy)-piperidine
4-(1H-imidazol-4-ylmethoxy)-1-[(5-methyl-2-thienyl)methyl]-piperidine
1-[(4-bromo-2-thienyl)methyl]-4-(1H-imidazol-4-ylmethoxy)-piperidine
1-([2,2′-bithiophen]-5-ylmethyl)-4-(1H-imidazol-4-ylmethoxy)-piperidine
3,5-dichloro-2-[[4-(1H-imidazol-4-ylmethoxy)-1-piperidinyl]methyl]-phenol
1-[(3,4-difluorophenyl)methyl]-4-(1H-imidazol-4-ylmethoxy)-piperidine
1-[(3,5-difluorophenyl)methyl]-4-(1H-imidazol-4-ylmethoxy)-piperidine
1-[(6-chloro-1,3-benzodioxol-5-yl)methyl]-4-(1H-imidazol-4-ylmethoxy)-piperidine
1-[[4-[4-(1,1-dimethylethyl)-2-thiazolyl]phenyl]methyl]-4-(1H-imidazol-4-ylmethoxy)-piperidine
4-(1H-imidazol-4-ylmethoxy)-1-[(1-methyl-1H-pyrrol-2-yl)methyl]-piperidine
1H-indole, 3-[[4-(1H-imidazol-4-ylmethoxy)-1-piperidinyl]methyl]-1-(phenylmethyl)
1-[(5-chloro-2-thienyl)methyl]-4-(1H-imidazol-4-ylmethoxy)-piperidine
1-(1,3-benzodioxol-4-ylmethyl)-4-(1H-imidazol-4-ylmethoxy)-piperidine
2-thiophenecarbonitrile, 3-[[4-[[4-(1H-imidazol-4-ylmethoxy)-1-piperidinyl]methyl]phenoxy]methyl]-piperidine
4-(1H-imidazol-4-ylmethoxy)-1-[[5-(phenylethynyl)-2-thienyl]methyl]-piperidine
4-(1H-imidazol-4-ylmethoxy)-1-[[5-(4-nitrophenyl)-2-furanyl]methyl]-piperidine
4-(1H-imidazol-4-ylmethoxy)-1-[[5-(3-nitrophenyl)-2-furanyl]methyl]-piperidine
1-[(4-chloro-1H-pyrazol-3-yl)methyl]-4-(1H-imidazol-4-ylmethoxy)-piperidine
1-[(4-bromo-1-methyl-1H-pyrazol-3-yl)methyl]-4-(1H-imidazol-4-ylmethoxy)-piperidine
1-[(4-bromo-1H-pyrazol-3-yl)methyl]-4-(1H-imidazol-4-ylmethoxy)-piperidine
2-[[4-(1H-imidazol-4-ylmethoxy)-1-piperidinyl]methyl]-benzonitrile
4-(1H-imidazol-4-ylmethoxy)-1-[(4-iodophenyl)methyl]-piperidine
1-[(5-ethyl-2-thienyl)methyl]-4-(1H-imidazol-4-ylmethoxy)-piperidine
4-(1H-imidazol-4-ylmethoxy)-1-[[5-(methylthio)-2-thienyl]methyl]-piperidine
1-[[1-(3,5-dichlorophenyl)-1H-pyrrol-2-yl]methyl]-4-(1H-imidazol-4-ylmethoxy)-piperidine
1-[[1-(4-chlorophenyl)-1H-pyrrol-2-yl]methyl]-4-(1H-imidazol-4-ylmethoxy)-piperidine
4-(1H-imidazol-4-ylmethoxy)-1-[[4-(phenylethynyl)-2-thienyl]methyl]-piperidine
4-(1H-imidazol-4-ylmethoxy)-1-[(3-phenoxy-2-thienyl)methyl]-piperidine
1-[[2-chloro-5-(trifluoromethyl)phenyl]methyl]-4-(1H-imidazol-4-ylmethoxy)-piperidine
4-(1H-imidazol-4-ylmethoxy)-1-[(4-propoxyphenyl)methyl]-piperidine
2-[[4-(1H-imidazol-4-ylmethoxy)-1-piperidinyl]methyl]-phenol
1-[(2,4-difluorophenyl)methyl]4-(1H-imidazol-4-ylmethoxy)-piperidine
3-[[4-(1H-imidazol-4-ylmethoxy)-1-piperidinyl]methyl]-2-thiophenecarbonitrile
1-(benzo[b]thien-3-ylmethyl)-4-(1H-imidazol-4-ylmethoxy)-piperidine
2-chloro-3-[[4-(1H-imidazol-4-ylmethoxy)-1-piperidinyl]methyl]-pyridine
3-[[4-(1H-imidazol-4-ylmethoxy)-1-piperidinyl]methyl]-2-(2-propenyl)-phenol
1-[(4-chloro-3-fluorophenyl)methyl]-4-(1H-imidazol-4-ylmethoxy)-piperidine
4-(1H-imidazol-4-ylmethoxy)-1-[[4-(trifluoromethoxy)phenyl]methyl]-piperidine
1-[(2,6-difluorophenyl)methyl]-4-(1H-imidazol-4-ylmethoxy)-piperidine
1-[(4-bromo-2-fluorophenyl)methyl]-4-(1H-imidazol-4-ylmethoxy)-piperidine
1-[(2,2-difluoro-1,3-benzodioxol-5-yl)methyl]-4-(1H-imidazol-4-ylmethoxy)-piperidine
1-[(4-butoxyphenyl)methyl]-4-(1H-imidazol-4-ylmethoxy)-piperidine
4-(1H-imidazol-4-ylmethoxy)-1-[(2,3,5-trichlorophenyl)methyl]-piperidine
1-[(2,5-dichlorophenyl)methyl]-4-(1H-imidazol-4-ylmethoxy)-piperidine
4-(1H-imidazol-4-ylmethoxy)-1-[[2-(trifluoromethyl)phenyl]methyl]-piperidine
1-[(4-chloro-2-nitrophenyl)methyl]-4-(1H-imidazol-4-ylmethoxy)-piperidine
   and pharmaceutically acceptable salts and solvates thereof.

The invention also comprises compounds according to formula (IA). In this aspect the invention therefore provides a compound of formula (IA): 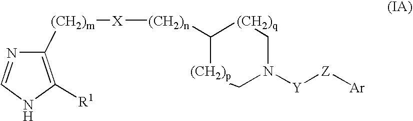
in which:

Ar is an aryl group, a 5-7 membered heteraromatic ring containing 1-4 heteroatoms selected from nitrogen, oxygen or sulphur, or a bicyclic or tricyclic heteraromatic ring containing 1-4 heteroatoms selected from nitrogen, oxygen or sulphur, each of which can be optionally substituted by 1-3 groups selected from C₁₋₆ alkyl, C₁₋₆ alkylthio, C₁₋₄ alkoxy, halogen, cyano, CF₃, OCF₃, C₃₋₆ cyclolalkyl, C₂₋₄ alkenyl, C₂₋₆ alkynyl, C₂₋₆ alkenyloxy, hydroxyl, nitro, tosyl, thienyl, benzyl, phenyl, nitrophenyl,

R¹ is hydrogen or C₁₋₄ alkyl;

X is O, NR², CH₂ or SOₓ

R² is C₁₋₆ alkyl;

x is 0, 1 or 2;

Y is C═O, SO₂, or (C═O)NH;

Z is (CR³R⁴), or Y and Z together form a CH═CH group;

m and n are independently 0, 1, 2 or 3;

p and q are independently 0, 1 or 2;

r is 0, 1, 2, 3, or 4, and

R³ and R⁴ are independently hydrogen or C₁₋₆alkyl.

For compounds (IA) Y is preferably C═O. Other preferred substituents for compounds of formula (IA) are those defined above.

According to another aspect of the invention there is also provided a process for the preparation of compounds (I)/(IA) which comprises:

(a) for compounds of formula (I) where Y is C═O, reaction of a compound of formula (II): 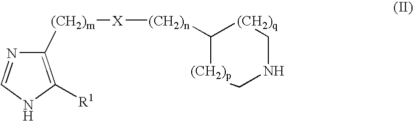
in which R¹, X, m, n, p and q are as defined in formula (I) or are protected derivatives thereof, with a compound of formula (III):
Ar—(CR³R⁴)ᵣ—CO₂H (III)

in which Ar, R³, R⁴ and r are as defined in formula (I) or are protected derivatives thereof, or

(b) for compounds of formula (I) where Y is SO₂, reaction of a compound of formula (II) with a compound of formula (IV):
Ar—(CR³R⁴)ᵣ—SO₂ (IV)

in which Ar, R³, R⁴ and r are as defined in formula (I) or are protected derivatives thereof, or

(c) for compounds of formula (I) where Y is CONH, reaction of a compound of formula (II) with a carbonyl source such as phosgene or triphosgene and an amine Ar—(CR³R⁴)ᵣ—NH₂ or by treating with an isocyanate Ar—(CR³R⁴)ᵣ—NCO, or

(d) for compounds of formula (I) where r is 0 and Y is CH₂, reaction of a compound of formula (II) with a compound ArCHO by reductive amination,

and optionally thereafter,

removing any protecting groups

forming a pharmaceutically acceptable salt.

The reaction between compounds (II) and (III) may be carried out using standard coupling conditions for example using peptide coupling reagents such as HOBt, DCC PyBrop, or via an acid chloride in the presence of a base such as triethylamine in an inert solvent.

Reaction of compounds (II) and (IV) can be carried out in the presence of a base such as triethylamine or pyridine in an aprotic solvent such as dichloromethane.

Process (d) can be carried out by reductive amination using reagents such as solid supported cyanoborohydride resin, catalytic acetic acid in aprotic solvent such as dichloromethane or NMP, or alternatively sodium triacetoxyborohydride in dichloromethane with catalytic acetic acid.

Compounds of formula (II) where X is NH₂ or SH may be prepared from compounds of formula (V) 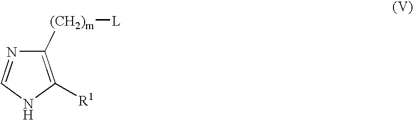
where R¹ and m are as defined above and L is a leaving group by reaction with a compound of formula (VI): 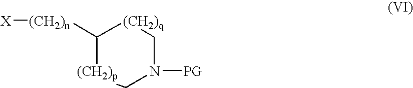
where X is NH₂ or SH, PG is a protecting group, L is a leaving group, and n, p and q and are as defined in formula (I), and where X is SH optionally oxidising the resulting compound of formula (II). The reaction can be carried out using an aprotic base such as thiethylamine or Hunig's base in a suitable solvent such as dichloromethane. Suitable protecting groups PG include acid labile groups such as tBoc. Compounds of formula (II) where X is S can be oxidised using oxone or mCPBA under controlled conditions to give the corresponding compounds where X is SO or SO₂.

Compounds of formula (II) where X is O can be prepared by reacting a compound of formula (V) as defined above with a compound of formula (VII): 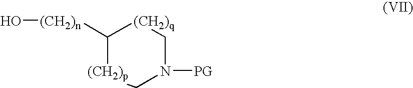
in which PG is a protecting group, L is a leaving group, and n, p and q and are as defined in formula (I). Preferably L in compound (V) is halide or a triflate, the reaction being carried out in the presence of a base such as sodium hydride or potassium t-butoxide. The group PG is an acid labile group such as t-Boc.

It will be appreciated by those skilled in the art that in the processes of the present invention certain functional groups such as hydroxyl or amino groups in the starting reagents or intermediate compound may need to be protected by protecting groups. Thus, the preparation of the compound of formula (I) may involve, at an appropriate stage, the removal of one or more protecting groups. The protection and deprotection of functional groups is fully described in ‘Protective Groups in Organic Chemistry’, edited by J. W. F. McOmie, Plenum Press (1973), and ‘Protective Groups in Organic Synthesis’, 2nd edition, T. W. Greene & P. G. M. Wuts, Wiley-Interscience (1991).

The compounds of formula (I) above may be converted to a pharmaceutically acceptable salt or solvate thereof, preferably a basic addition salt such as sodium, potassium, calcium, aluminium, lithium, magnesium, zinc, benzathine, chloroprocaine, choline, diethanolamine, ethanolamine, ethyldiamine, meglumine, tromethamine or procaine, or an acid addition salt such as a hydrochloride, hydrobromide, phosphate, acetate, fumarate, maleate, tartrate, citrate, oxalate, methanesulphonate or p-toluenesulphonate.

The compounds of formula (I) have activity as pharmaceuticals, in particular as modulators of histamine H4, and may be used in the treatment (therapeutic or prophylactic) of conditions/diseases in human and non-human animals including:

(1) (the respiratory tract) obstructive airways diseases including chronic obstructive pulmonary disease (COPD); asthma, such as bronchial, allergic, intrinsic, extrinsic and dust asthma, particularly chronic or inveterate asthma (e.g. late asthma and airways hyper-responsiveness); bronchitis; acute, allergic, atrophic rhinitis and chronic rhinitis including rhinitis caseosa, hypertrophic rhinitis, rhinitis purulenta, rhinitis sicca and rhinitis medicamentosa; membranous rhinitis including croupous, fibrinous and pseudomembranous rhinitis and scrofoulous rhinitis; seasonal rhinitis including rhinitis nervosa (hay fever) and vasomotor rhinitis; sarcoidosis, farmer's lung and related diseases, fibroid lung and idiopathic interstitial pneumonia;

(2) (bone and joints) gout, rheumatoid arthritis, seronegative spondyloarthropathies (including ankylosing spondylitis, psoriatic arthritis and Reiter's disease), Behcet's disease, Sjogren's syndrome and systemic sclerosis;

(3) (skin) pruritis, scleroderma, otitus, psoriasis, atopical dermatitis, contact dermatitis and other eczmatous dermitides, seborrhoetic dermatitis, Lichen planus, Pemphigus, bullous Pemphigus, Epidermolysis bullosa, urticaria, angiodermas, vasculitides, erythemas, cutaneous eosinophilias, uveitis, Alopecia areata and vernal conjunctivitis, lupus;

(4) (gastrointestinal tract) Coeliac disease, proctitis, eosinopilic gastro-enteritis, mastocytosis, inflammatory bowel diseases such as Crohn's disease, ulcerative colitis, ileitis and enteritis, food-related allergies which have effects remote from the gut, e.g., migraine, rhinitis and eczema;

(5) (central and peripheral nervous system) Neurodegenerative diseases and dementia disorders, e.g. Alzheimer's disease, amyotrophic lateral sclerosis and other motor neuron diseases, Creutzfeldt-Jacob's disease and other prion diseases, HIV encephalopathy (AIDS dementia complex), Huntington's disease, frontotemporal dementia, Lewy body dementia and vascular dementia; polyneuropathies, e.g. Guillain-Barré syndrome, chronic inflammatory demyelinating polyradiculoneuropathy, multifocal motor neuropathy, plexopathies; CNS demyelination, e.g. multiple sclerosis, acute disseminated/haemorrhagic encephalomyelitis, and subacute sclerosing panencephalitis; neuromuscular disorders, e.g. myasthenia gravis and Lambert-Eaton syndrome; spinal disorders, e.g. tropical spastic paraparesis, and stiff-man syndrome: paraneoplastic syndromes, e.g. cerebellar degeneration and encephalomyelitis; CNS trauma; migraine; stroke and correctum diseases such as meningitis

(6) (other tissues and systemic disease) hepatitis, vasculitis, spondyloarthopathies, vaginitis, glomerulonephritis, myositis, atherosclerosis, Acquired Immunodeficiency Syndrome (AIDS), lupus erythematosus, systemic lupus, erythematosus, Hashimoto's thyroiditis, type I diabetes, nephrotic syndrome, eosinophilia fascitis, hyper IgE syndrome, lepromatous leprosy, and idiopathic thrombocytopenia pupura; post-operative adhesions, and sepsis.

(7) (allograft and xenograft rejection) acute and chronic following, for example, transplantation of kidney, heart, liver, lung, bone marrow, skin and cornea; and chronic graft versus host disease;

(8) (cancer, carcinoma and tumour metastasis) including that of the bladder, breast, colon, kidney, liver, lung, ovary, pancreas, stomach, cervix, thyroid and skin, especially non-small cell lung cancer (NSCLC), malignant melanoma, prostate cancer and squamous sarcoma. Hematopoietic tumors of lymphoid lineage, including acute lymphocytic leukemia, B cell lymphoma and Burketts lymphoma, Hodgkins Lymphoma, Acute Lymphoblastic Leukemia. Hematopoietic tumors of myeloid lineage, including acute and chronic myelogenous leukemias and promyelocytic leukemia. Tumors of mesenchymal origin, including fibrosarcoma and rhabdomyosarcoma, and other tumors, including melanoma, seminoma, tetratocarcinoma, neuroblastoma and glioma.

(9) All diseases that result from a general inbalance of the immune system and resulting in increased atopic inflammatory reactions.

(10) Cystic fibrosis, re-perfusion injury in the heart, brain, peripheral limbs and other organs.

(11) Burn wounds & chronic skin ulcers

(12) Reproductive Diseases (e.g. Disorders of ovulation, menstruation and implantation, Pre-term labour, Endometriosis)

(13) thrombosis

(14) infectious diseases such as HIV infection and other viral infections, bacterial infections.

Thus, the present invention provides a compound of formula (IA), or a pharmaceutically-acceptable salt or solvate thereof, as hereinbefore defined for use in therapy.

Preferably the compounds of the invention are used to treat respiratory diseases. It is preferred that the compound of the invention is used to treat asthma and rhinitis, especially asthma.

In a further aspect, the present invention provides the use of a compound of formula (IA), or a pharmaceutically acceptable salt or solvate thereof, as hereinbefore defined in the manufacture of a medicament for use in therapy.

In a still further aspect, the present invention provides the use of a compound of formula (IA), or a pharmaceutically acceptable salt or solvate thereof, as hereinbefore defined in the manufacture of a medicament for the treatment of human diseases or conditions in which modulation of histamine H4 receptor activity is beneficial.

In the context of the present specification, the term “therapy” also includes “prophylaxis” unless there are specific indications to the contrary. The terms “therapeutic” and “therapeutically” should be construed accordingly.

The invention still further provides a method of treating a histamine H4 mediated disease, which comprises administering to a patient a therapeutically effective amount of a compound of formula (I) or (IA), or a pharmaceutically acceptable salt or solvate thereof, as hereinbefore defined.

The invention also provides a method of treating a respiratory disease, such as asthma and rhinitis, especially asthma, in a patient suffering from, or at risk of, said disease, which comprises administering to the patient a therapeutically effective amount of a compound of formula (I) or (IA), or a pharmaceutically acceptable salt or solvate thereof, as hereinbefore defined.

For the above-mentioned therapeutic uses the dosage administered will, of course, vary with the compound employed, the mode of administration, the treatment desired and the disorder indicated.

The compound of formula (I) and pharmaceutically acceptable salts and solvates thereof may be used on their own but will generally be administered in the form of a pharmaceutical composition in which the formula (I) compound/salt/solvate (active ingredient) is in association with a pharmaceutically acceptable adjuvant, diluent or carrier. Depending on the mode of administration, the pharmaceutical composition will preferably comprise from 0.05 to 99% w (percent by weight), more preferably from 0.05 to 80% w, still more preferably from 0.10 to 70% w, and even more preferably from 0.10 to 50% w, of active ingredient, all percentages by weight being based on total composition.

The present invention also provides a pharmaceutical composition comprising a compound of formula (IA), or a pharmaceutically acceptable salt or solvate thereof, as hereinbefore defined, in association with a pharmaceutically acceptable adjuvant, diluent or carrier.

The invention further provides a process for the preparation of a pharmaceutical composition of the invention which comprises mixing a compound of formula (IA), or a pharmaceutically acceptable salt or solvate thereof, as hereinbefore defined, with a pharmaceutically acceptable adjuvant, diluent or carrier.

The compounds of the invention can be administered in combination with other agents such as long-acting β-agonists.

The pharmaceutical compositions may be administered topically (e.g. to the lung and/or airways or to the skin) in the form of solutions, suspensions, heptafluoroalkane aerosols and dry powder formulations; or systemically, e.g. by oral administration in the form of tablets, capsules, syrups, powders or granules, or by parenteral administration in the form of solutions or suspensions, or by subcutaneous administration or by rectal administration in the form of suppositories or transdermally. Preferably the compound of the invention is administered orally.

The following examples illustrate the invention.

Preparation of Intermediates

### 1-Piperidinecarboxylic acid, 4-[[1-(triphenylmethyl)-1H-imidazol-4-yl]methoxy]-1,1-dimethylethyl ester

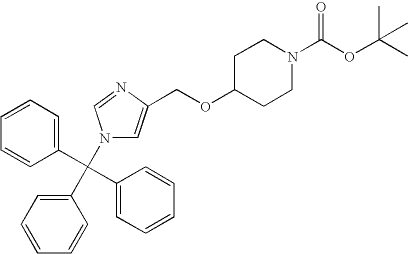

To a solution of 4-hydroxy-1-piperidinecarboxylic acid, 1,1-dimethylethyl ester (9.5 g, 0.047 mol) in dry N-methylpyrrollidine (NMP) (50 ml) was added sodium hydride (60% in oil) portionwise (1.88 g, 0.047 mol). The mixture was allowed to stir for 30 minutes, then 4-(chloromethyl)-1-(triphenylmethyl)-1H-imidazole (ref: WO0244141) (16.8 g, 0.047 mol) was added and the mixture heated to 50° C. for 30 min, poured into ice water and extracted with ethyl acetate. The organic extacts were washed with water and purified by flash column chromatography eluting with 1% methanolic ammonia/dichloromethane to give a solid (5.5 g). 300 MHz 1H NMR (CDCl₃) 7.41 (1H, d), 7.32-7.13 (15H, m), 6.81 (1H, bs), 4.49 (2H, s), 3.82-3.77 (2H, m), 3.62-3.54 (1H, m), 3.05-2.96 (2H, m), 1.85-1.76 (2H, m), 1.57-1.48 (2H, m), 1.45 (9H, s)

### 1-Piperidinecarboxylic acid, 4-[[5-methyl-1-(triphenylmethyl)-1H-imidazol-4-yl]methoxy]-1,1-dimethylethyl ester

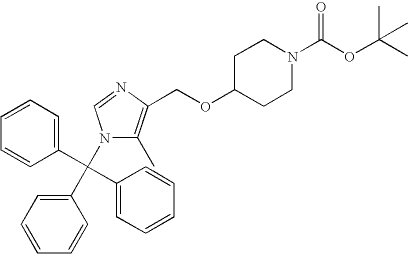

This was prepared by the method of Example 1 from 4-(chloromethyl)-5-methyl-1-(triphenylmethyl)-1H-imidazole (ref: European Journal of Medicinal Chemistry (1990), 25(7), 557). 300 MHz 1H NMR (CDCl₃) 7.34-7.29 (15H, m), 7.26 (1H, d), 4.47 (2H, s), 3.82-3.77 (2H, m), 3.62-3.54 (1H, m), 3.05-2.96 (2H, m), 2.25 (3H, s), 1.85-1.76 (2H, m), 1.57-1.48 (2H, m), 1.45 (9H, s)

### 4-[[1-(Triphenylmethyl)-1H-imidazol-4-yl]methoxy]-piperidine

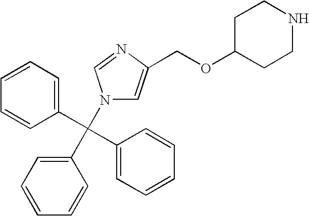

Trimethylsilyliodide (3.3 ml, 1 eq.) was added to a solution of 1-piperidinecarboxylic acid, 4-[[1-(triphenylmethyl)-1H-imidazol-4-yl]methoxy]-1,1-dimethylethyl ester (12 g, 0.023 mol) at 0-5° C., stirred at this temperature for 30 minutes, quenched with ice cold sodium bicarbonate solution and the organic layer separated, dried over sodium sulphate and evaporated. The residue was purified by flash column chromatography eluting with 5% methanolic ammonia/dichloromethane to give a solid (6.9 g). 300 MHz 1H NMR (CDCl₃) 7.41 (1H, d), 7.11-7.39 (15H, m), 6.81 (1H, d), 4.48 (2H, s), 3.53 (1H, m), 3.41 (2H, s), 3.08 (2H, m), 2.67 (2H, m), 1.99 (2H, m), 1.51 (2H, m)

### 4-[[5-Methyl-1-(triphenylmethyl)-1H-imidazol-4-yl]methoxy]-piperidine

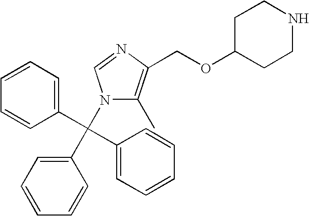 This was prepared by the method of example 2. MS (+APCI) m/z 438 (M+H⁺)
Preparation of Final Products

### EXAMPLE 1

### 4-(1H-Imidazol-4-ylmethoxy)-1-(1-oxo-3-phenylbutyl)-piperidine

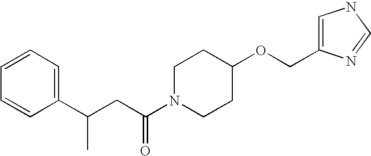

Bromo-tris-pyrrolidinophosphonium hexafluorophosphate (PyBrop™) (0.55 g, 1.18 mmol) was added to a solution of 4-[[1-(triphenylmethyl)-1H-imidazol-4-yl]methoxy]-piperidine (0.5 g, 1.18 mmol) and 3-phenylbutanoic acid (0.24 g, 1.18 mmol), Hunig's base (1 ml) in dry NMP. The mixture was stirred at room temp for 16 h, evaporated and the residue dissolved in methanol, filtered through sulphonic acid ion exchange resin eluting with methanol followed by methanolic ammonia, and evaporated. The residue was purified by reverse phase HPLC on an Xterra™ column eluting with acetonitrile/aqueous ammonium hydroxide to give the product as a white solid (0.052 g). MS (+APCI) m/z 328 (M+H⁺). 400 MHz 1H NMR (d6-DMSO) (at 125° C.—spectrum at room temperature complicated due to rotamers) 7.54 (1H, s), 7.29-7.24 (3H, m), 7.19-7.14 (2H, m), 6.91 (1H, s), 4.42 (2H, s), 3.75-3.58 (2H, m), 3.30-3.08 (2H, m), 2.8 (1H, br.s), 2.61 (2H, dd), 2.53 (4H dd), 1.80-1.70 (2H, m), 1.47-1.33 (H, m), 1.25 (2H, d)

### EXAMPLE 2

### 4-(1H-Imidazol-4-ylmethoxy)-1-[[4-(trifluoromethyl)phenyl]acetyl]-piperidine

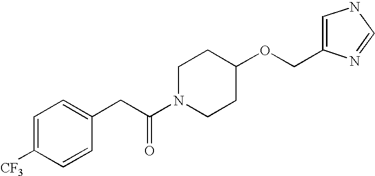

The title compound was prepared using the method of Example 1 with [4-(trifluoromethyl)phenyl]acetic acid: MS (+APCI) m/z 368 (M+H⁺). 400 MHz 1H NMR (d6-DMSO) (at 125° C.—spectrum at room temperature complicated due to rotamers) 7.63 (2H, d), 7.57 (1H, s), 7.44 (2H, d), 7.02 (1H, br.s), 4.44 (2H, s), 3.83 (2H, s), 3.80-3.73 (1H, m), 3.70-3.62 (2H, m), 3.31-3.22 (2H, m), 2.88 (2H, br.s), 1.83-1.73 (2H, br.m), 1.50-1.41 (2H, br.m)

### EXAMPLE 3

### 1-[2-(4-Hydroxyphenyl)-1-oxopropyl]-4-[(5-methyl-1H-imidazol-4-yl)methoxy]-piperidine

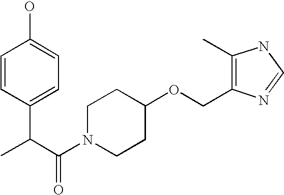

This was prepared by the method of Example 1 using 2-(4-hydroxyphenyl)propanoic acid and 4-[[5-methyl-1-(triphenylmethyl)-1H-imidazol-4-yl]methoxy]-piperidine. MS (+APCI) m/z 344 (M+H⁺). 400 MHz 1H NMR (d6-DMSO) 11.45 (1H, s), 8.96 (1H, s), 7.32 (1H, s), 6.96 (2H, m), 6.68 (2H, m), 4.30 (2H, s), 3.93 (1H, s), 3.71 (2H, m), 3.46 (1H, m), 3.25 (2H, m), 2.09 (3H, s), 1.63 (2H, m), 1.23 (5H, m)

The compounds in Table 1 were prepared using the method of Example 1 with the appropriate acid

TABLE 1

| | Example | | M + H⁺ |
|---|---|---|---|
| Example structure | no. | Name | (+APCI) |
| 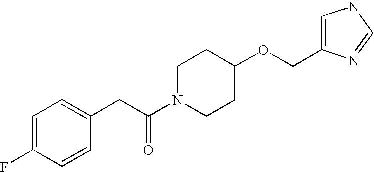 | 4 | piperidine, 1-[(4- fluorophenyl)acetyl]-4-(1H- imidazol-4-ylmethoxy)- | 317 |
| 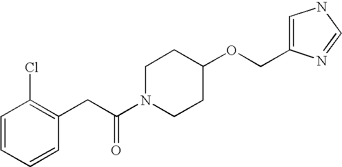 | 5 | piperidine, 1-[(2- chlorophenyl)acetyl]-4-(1H- imidazol-4-ylmethoxy)- | 333 |
| 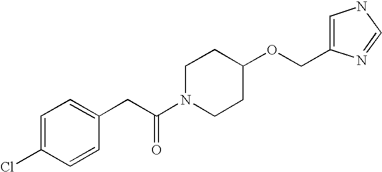 | 6 | piperidine, 1-[(4- chlorophenyl)acetyl]-4-(1H- imidazol-4-ylmethoxy)- | 333 |
| 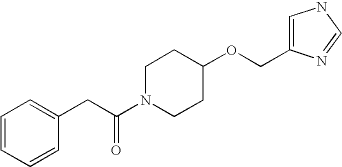 | 7 | piperidine, 4-(1H-imidazol-4- ylmethoxy)-1-(phenylacetyl)- | 299 |
| 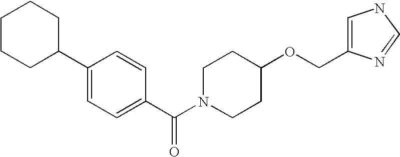 | 8 | piperidine, 1-(4- cyclohexylbenzoyl)-4-(1H- imidazol-4-ylmethoxy)- | 367 |
| 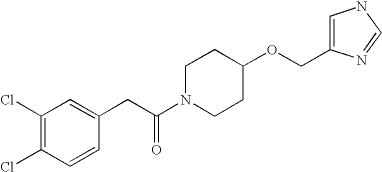 | 9 | piperidine, 1-[(3,4- dichlorophenyl)acetyl]-4- (1H-imidazol-4-ylmethoxy)- | 367 |
| 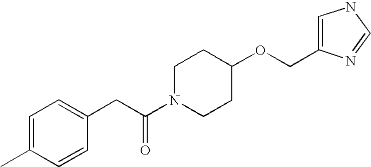 | 10 | piperidine, 4-(1H-imidazol-4- ylmethoxy)-1-[(4- methylphenyl)acetyl]- | 311 |
| 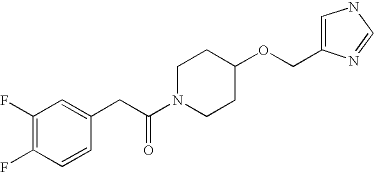 | 11 | piperidine, 1-[(3,4- difluorophenyl)acetyl]-4-(1H- imidazol-4-ylmethoxy)- | 327 |
| 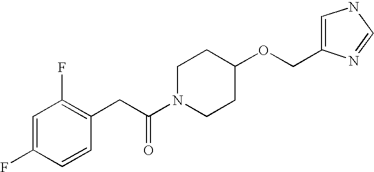 | 12 | piperidine, 1-[(2,4- difluorophenyl)acetyl]-4-(1H- imidazol-4-ylmethoxy)- | 317 |
| 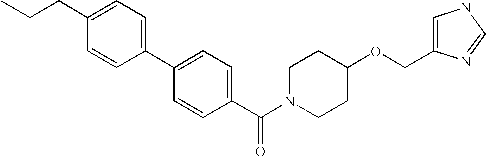 | 13 | piperidine, 4-(1H-imidazol-4- ylmethoxy)-1-[(4′- propyl[1,1′-biphenyl]-4- yl)carbonyl]- | 355 |
| 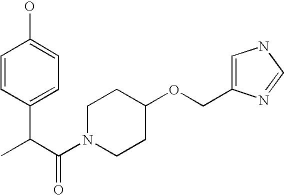 | 14 | piperidine, 1-[2-(4- hydroxyphenyl)-1- oxopropyl]-4-(1H-imidazol-4- ylmethoxy)- | 329 |
| 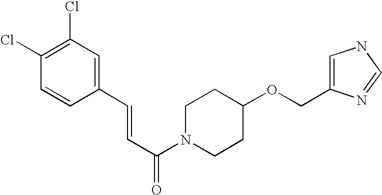 | 15 | piperidine, 1-[(2E)-3-(3,4- dichlorophenyl)-1-oxo-2- propenyl]-4-(1H-imidazol-4- ylmethoxy)- | 379 |
| 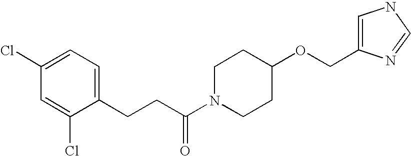 | 16 | piperidine, 1-[3-(2,4- dichlorophenyl)-1- oxopropyl]-4-(1H-imidazol-4- ylmethoxy)- | 381 |
| 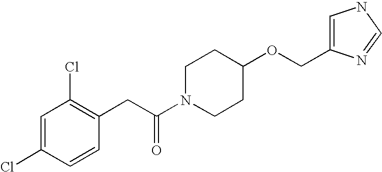 | 17 | piperidine, 1-[(2,4- dichlorophenyl)acetyl]-4- (1H-imidazol-4-ylmethoxy)- | 367 |

### EXAMPLE 18

### 1-[(2-Bromophenyl)methyl]-4-(1H-imidazol-4-ylmethoxy)-piperidine

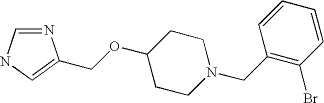 To a solution of 4-[[1-(triphenylmethyl)-1H-imidazol-4-yl]methoxy]-piperidine (0.5 g, 1.18 mmol) and 2-bromobenzaldehyde (0.218 g, 1.18 mmol) in 10% acetic acid/N-methylpyrrollidine (10 ml) was added (polystyrylmethyl)trimethylammonium cyanoborohydride resin (4.2 mmol/g equivalent, 300 mg). The mixture was stirred 16 h at room temperature, the resin removed by filtration and the solution evaporated. The residue was purified by reserve phase HPLC on an Xterra™ column using aqueous ammonium acetate/acetonitrile as eluant to give the title compound (0.058 g). MS (+APCI) m/z 350 (M+H⁺). 400 MHz ¹H NMR (d6-DMSO) 7.59-7.56 (2H, m), 7.46 (1H, d), 7.36 (1H, t), 7.19 (1H, t), 7.05-6.8 (2H, br.m), 4.37 (2H, br.s), 3.50 (2H, s), 3.40 (1H, br.s), 2.71-2.66 (2H, m), 2.18-2.12 (2H, m), 1.84-1.81 (2H, m), 1.50-1.41 (2H, m)

### EXAMPLE 19

### 1-[(3-Bromo-2-thienyl)methyl]-4-[(5-methyl-1H-imidazol-4-yl)methoxy]-piperidine

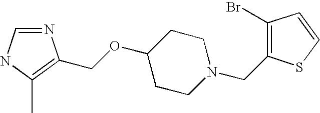

This was prepared by the method of Example 18 using 3-bromothiophene-2-carboxaldehyde and 4-[[5-methyl-1-(triphenylmethyl)-1H-imidazol-4-yl]methoxy]-piperidine. MS (+APCI) m/z 372 (M+H⁺). 400 MHz 1H NMR (d6-DMSO) 7.57 (1H, d, J=5.4 Hz), 7.40 (1H, s), 7.02 (1H, d, J=5.4 Hz), 4.32 (2H, s), 3.61 (2H, s), 3.29 (1H, m), 2.73 (2H, m), 2.17 (21H, m), 2.11 (3H, s), 1.82 (2H, m), 1.42 (2H, m)

The compounds in Table 2 were prepared using the method of Example 18 with the appropriate acid

TABLE 2

| | Example | | M + H⁺ |
|---|---|---|---|
| Example structure | no. | Name | (+APCI) |
| 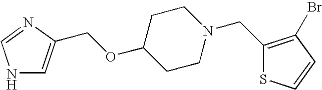 | 20 | piperidine, 1-[(3- bromo-2- thienyl)methyl]-4- (1H-imidazol-4- ylmethoxy)- | 355 |
| 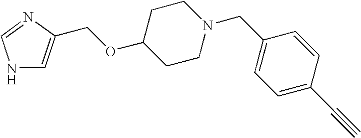 | 21 | piperidine, 1-[(4- ethynylphenyl)methyl]- 4-(1H-imidazol-4- ylmethoxy)- | 295 |
| 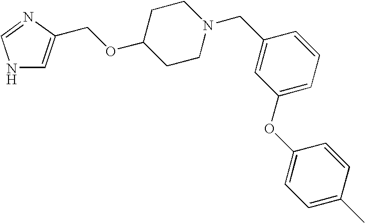 | 22 | piperidine, 4-(1H- imidazol-4- ylmethoxy)-1-[[3-(4- methylphenoxy)- phenyl]methyl]- | 377 |
| 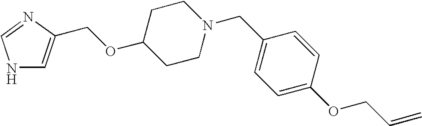 | 23 | piperidine, 4-(1H- imidazol-4- ylmethoxy)-1-[[4-(2- propenyloxy)phenyl]methyl]- | 327 |
| 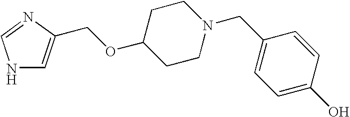 | 24 | phenol, 4-[[4-(1H- imidazol-4- ylmethoxy)-1- piperidinyl]methyl]- | 287 |
| 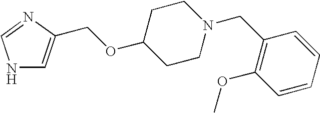 | 25 | piperidine, 4-(1H- imidazol-4- ylmethoxy)-1-[(2- methoxyphenyl)- methyl]- | 301 |
| 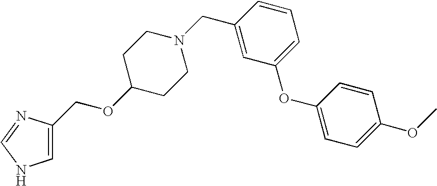 | 26 | piperidine, 4-(1H- imidazol-4- ylmethoxy)-1-[[3-(4- methoxyphenoxy)- phenyl]methyl]- | 393 |
| 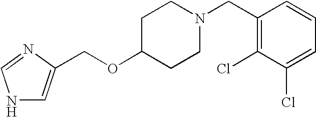 | 27 | piperidine, 1-[(2,3- dichlorophenyl)- methyl]-4-(1H-imidazol-4- ylmethoxy)- | 339 |
| 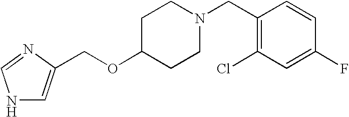 | 28 | piperidine, 1-[(2- chloro-4- fluorophenyl)methyl]- 4-(1H-imidazol-4- ylmethoxy)- | 323 |
| 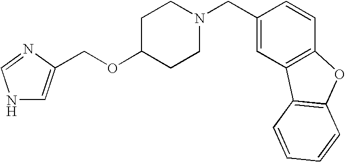 | 29 | piperidine, 1-(2- dibenzofuranylmethyl)- 4-(1H-imidazol-4- ylmethoxy)- | 361 |
| 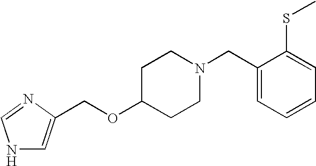 | 30 | piperidine, 4-(1H- imidazol-4- ylmethoxy)-1-[[2- (methylthio)phenyl]- methyl]- | 317 |
| 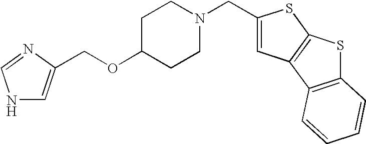 | 31 | piperidine, 4-(1H- imidazol-4- ylmethoxy)-1- (thieno[2,3- b][1]benzothien-2- ylmethyl)- | 383 |
| 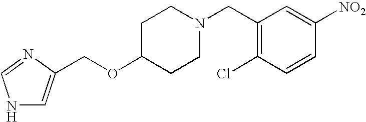 | 32 | piperidine, 1-[(2- chloro-5- nitrophenyl)methyl]- 4-(1H-imidazol-4- ylmethoxy)- | 350 |
| 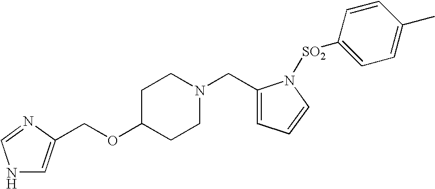 | 33 | 1H-pyrrole, 2-[[4-(1H- imidazol-4- ylmethoxy)-1- piperidinyl]methyl]-1- [(4- methylphenyl)sulfonyl]- | 414 |
| 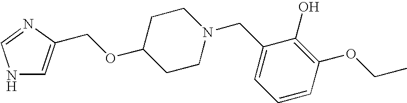 | 34 | phenol, 2-ethoxy-6- [[4-(1H-imidazol-4- ylmethoxy)-1- piperidinyl]methyl]- | 331 |
| 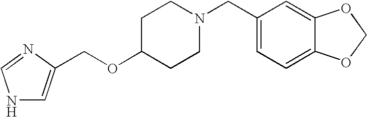 | 35 | piperidine, 1-(1,3- benzodioxol-5- ylmethyl)-4-(1H- imidazol-4- ylmethoxy)- | 315 |
| 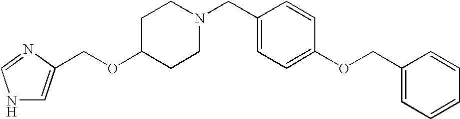 | 36 | piperidine, 4-(1H- imidazol-4- ylmethoxy)-1-[[4- (phenylmethoxy)- phenyl]methyl]- | 377 |
| 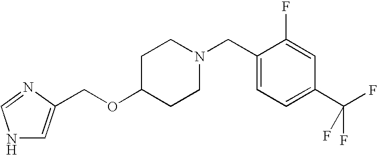 | 37 | piperidine, 1-[[2- fluoro-4- (trifluoromethyl)phenyl]- methyl]-4-(1H- imidazol-4- ylmethoxy)- | 357 |
| 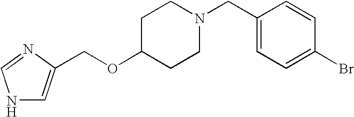 | 38 | piperidine, 1-[(4- bromophenyl)methyl]- 4-(1H-imidazol-4- ylmethoxy)- | 349 |
| 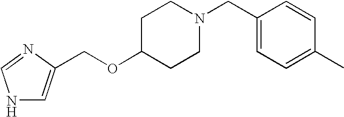 | 39 | piperidine, 4-(1H- imidazol-4- ylmethoxy)-1-[(4- methylphenyl)methyl]- | 285 |
| 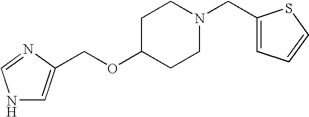 | 40 | piperidine, 4-(1H- imidazol-4- ylmethoxy)-1-(2- thienylmethyl)- | 277 |
| 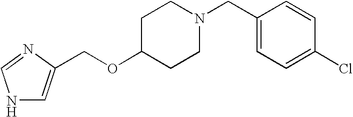 | 41 | piperidine, 1-[(4- chlorophenyl)methyl]- 4-(1H-imidazol-4- ylmethoxy)- | 305 |
| 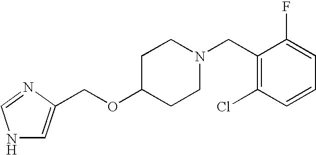 | 42 | piperidine, 1-[(2- chloro-6- fluorophenyl)methyl]- 4-(1H-imidazol-4- ylmethoxy)- | 323 |
| 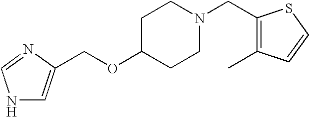 | 43 | piperidine, 4-(1H- imidazol-4- ylmethoxy)-1-[(3- methyl-2- thienyl)methyl]- | 291 |
| 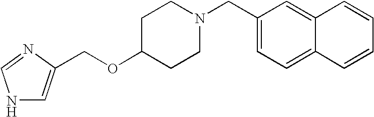 | 44 | piperidine, 4-(1H- imidazol-4- ylmethoxy)-1-(2- naphthalenylmethyl)- | 321 |
| 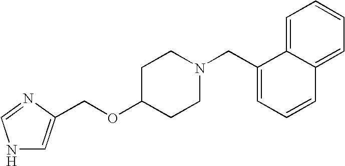 | 45 | piperidine, 4-(1H- imidazol-4- ylmethoxy)-1-(1- naphthalenylmethyl)- | 321 |
| 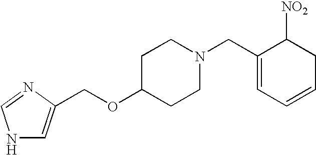 | 46 | piperidine, 4-(1H- imidazol-4- ylmethoxy)-1-[(2- nitrophenyl)methyl]- | 316 |
| 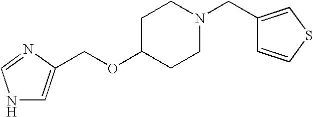 | 47 | piperidine, 4-(1H- imidazol-4- ylmethoxy)-1-(3- thienylmethyl)- | 277 |
| 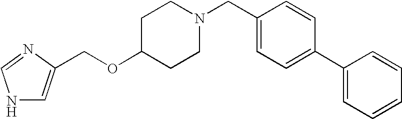 | 48 | piperidine, 1-([1,1′- biphenyl]-4- ylmethyl)-4-(1H- imidazol-4- ylmethoxy)- | 347 |
| 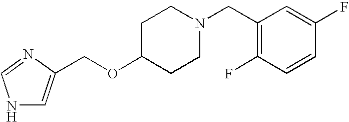 | 49 | piperidine, 1-[(2,5- difluorophenyl)methyl]- 4-(1H-imidazol-4- ylmethoxy)- | 307 |
| 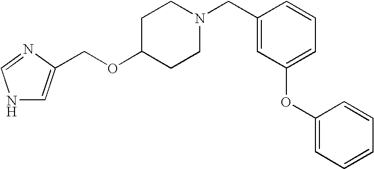 | 50 | piperidine, 4-(1H- imidazol-4- ylmethoxy)-1-[(3- phenoxyphenyl)- methyl]- | 363 |
| 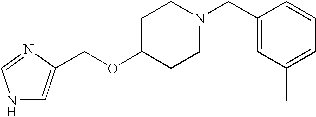 | 51 | piperidine, 4-(1H- imidazol-4- ylmethoxy)-1-[(3- methylphenyl)methyl]- | 285 |
| 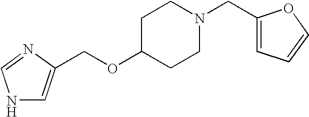 | 52 | piperidine, 1-(2- furanylmethyl)-4-(1H- imidazol-4- ylmethoxy)- | 261 |
| 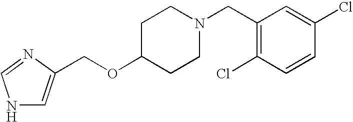 | 53 | piperidine, 1-[(2,6- dichlorophenyl)methyl]- 4-(1H-imidazol-4- ylmethoxy)- | 339 |
| 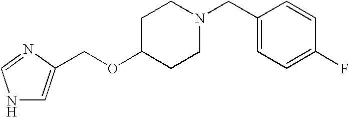 | 54 | piperidine, 1-[(4- fluorophenyl)methyl]- 4-(1H-imidazol-4- ylmethoxy)- | 289 |
| 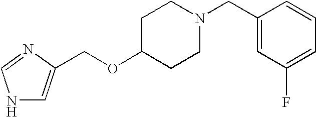 | 55 | piperidine, 1-[(3- fluorophenyl)methyl]- 4-(1H-imidazol-4- ylmethoxy)- | 289 |
| 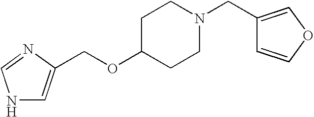 | 56 | piperidine, 1-(3- furanylmethyl)-4-(1H- imidazol-4- ylmethoxy)- | 261 |
| 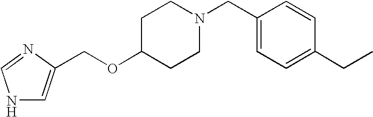 | 57 | piperidine, 1-[(4- ethylphenyl)methyl]- 4-(1H-imidazol-4- ylmethoxy)- | 299 |
| 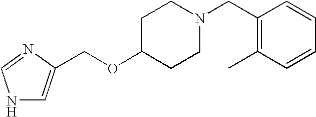 | 58 | piperidine, 4-(1H- imidazol-4- ylmethoxy)-1-[(2- methylphenyl)methyl]- | 285 |
| 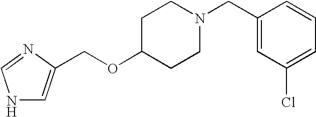 | 59 | piperidine, 1-[(3- chlorophenyl)methyl]- 4-(1H-imidazol-4- ylmethoxy)- | 305 |
| 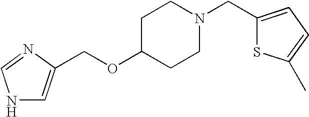 | 60 | piperidine, 4-(1H- imidazol-4- ylmethoxy)-1-[(5- methyl-2- thienyl)methyl]- | 291 |
| 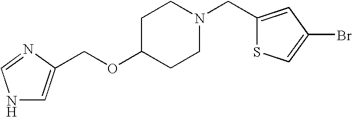 | 61 | piperidine, 1-[(4- bromo-2- thienyl)methyl]-4- (1H-imidazol-4- ylmethoxy)- | 355 |
| 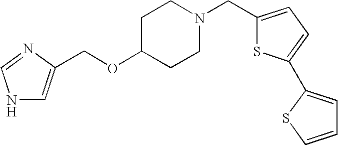 | 62 | piperidine, 1-([2,2′- bithiophen]-5- ylmethyl)-4-(1H- imidazol-4- ylmethoxy)- | 359 |
| 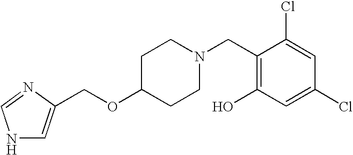 | 63 | phenol, 3,5-dichloro- 2-[[4-(1H-imidazol-4- ylmethoxy)-1- piperidinyl]methyl]- | 355 |
| 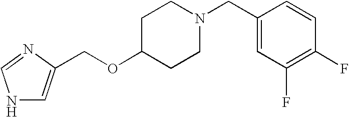 | 64 | piperidine, 1-[(3,4- difluorophenyl)methyl]- 4-(1H-imidazol-4- ylmethoxy)- | 307 |
| 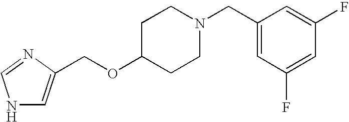 | 65 | piperidine, 1-[(3,5- difluorophenyl)methyl]- 4-(1H-imidazol-4- ylmethoxy)- | 307 |
| 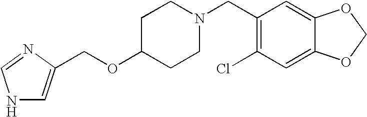 | 66 | piperidine, 1-[(6- chloro-1,3- benzodioxol-5- yl)methyl]-4-(1H- imidazol-4- ylmethoxy)- | 349 |
| 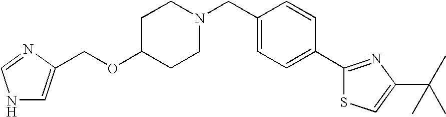 | 67 | piperidine, 1-[[4-[4- (1,1-dimethylethyl)-2- thiazolyl]phenyl]methyl]- 4-(1H-imidazol-4- ylmethoxy)- | 410 |
| 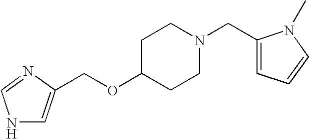 | 68 | piperidine, 4-(1H- imidazol-4- ylmethoxy)-1-[(1- methyl-1H-pyrrol-2- yl)methyl]- | 274 |
| 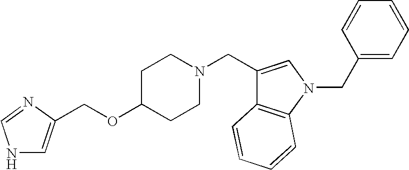 | 69 | 1H-indole, 3-[[4-(1H- imidazol-4- ylmethoxy)-1- piperidinyl]methyl]-1- (phenylmethyl)- | 400 |
| 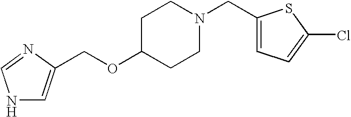 | 70 | piperidine, 1-[(5- chloro-2- thienyl)methyl]-4- (1H-imidazol-4- ylmethoxy)- | 311 |
| 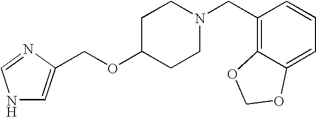 | 71 | piperidine, 1-(1,3- benzodioxol-4- ylmethyl)-4-(1H- imidazol-4- ylmethoxy)- | 315 |
| 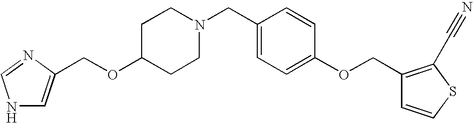 | 72 | 2- thiophenecarbonitrile, 3-[[4-[[4-(1H- imidazol-4- ylmethoxy)-1- piperidinyl]methyl]- phenoxy]methyl]- | 408 |
| 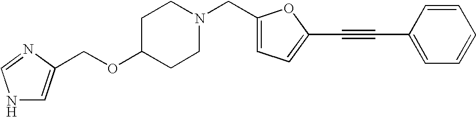 | 73 | piperidine, 4-(1H- imidazol-4- ylmethoxy)-1-[[5- (phenylethynyl)-2- thienyl]methyl]- | 377 |
| 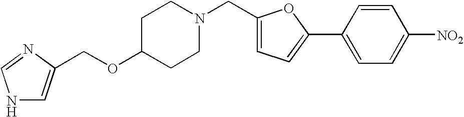 | 74 | piperidine, 4-(1H- imidazol-4- ylmethoxy)-1-[[5-(4- nitrophenyl)-2- furanyl]methyl]- | 382 |
| 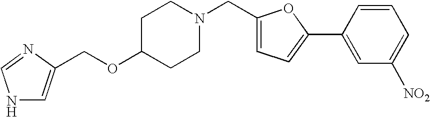 | 75 | piperidine, 4-(1H- imidazol-4- ylmethoxy)-1-[[5-(3- nitrophenyl)-2- furanyl]methyl]- | 382 |
| 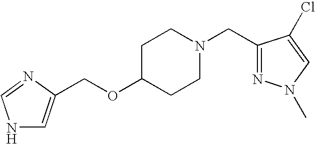 | 76 | piperidine, 1-[(4- chloro-1H-pyrazol-3- yl)methyl]-4-(1H- imidazol-4- ylmethoxy)- | 295 |
| 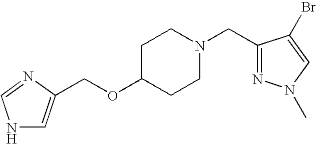 | 77 | piperidine, 1-[(4- bromo-1-methyl-1H- pyrazol-3-yl)methyl]- 4-(1H-imidazol-4- ylmethoxy)- | 353 |
| 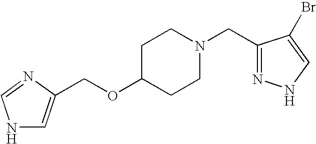 | 78 | piperidine, 1-[(4- bromo-1H-pyrazol-3- yl)methyl]-4-(1H- imidazol-4- ylmethoxy)- | 339 |
| 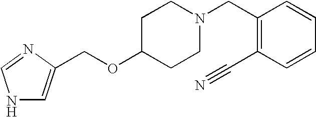 | 79 | benzonitrile, 2-[[4- (1H-imidazol-4- ylmethoxy)-1- piperidinyl]methyl]- | 296 |
| 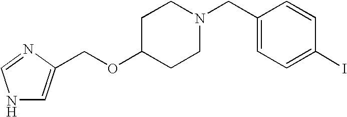 | 80 | piperidine, 4-(1H- imidazol-4- ylmethoxy)-1-[(4- iodophenyl)methyl]- | 397 |
| 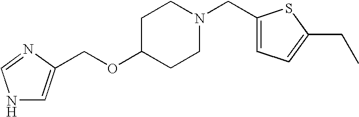 | 81 | piperidine, 1-[(5- ethyl-2- thienyl)methyl]-4- (1H-imidazol-4- ylmethoxy)- | 305 |
| 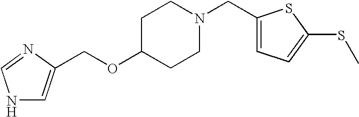 | 82 | piperidine, 4-(1H- imidazol-4- ylmethoxy)-1-[[5- (methylthio)-2- thienyl]methyl]- | 323 |
| 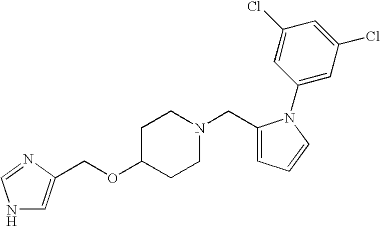 | 83 | piperidine, 1-[[1-(3,5- dichlorophenyl)-1H- pyrrol-2-yl]methyl]-4- (1H-imidazol-4- ylmethoxy)- | 404 |
| 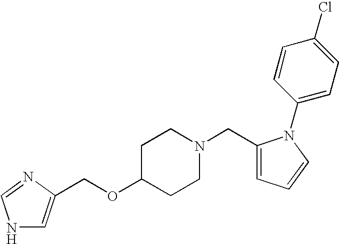 | 84 | piperidine, 1-[[1-(4- chlorophenyl)-1H- pyrrol-2-yl]methyl]-4- (1H-imidazol-4- ylmethoxy)- | 370 |
| 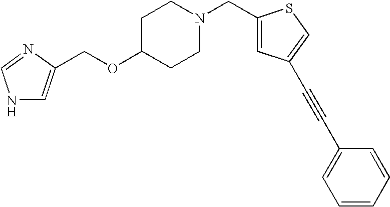 | 85 | piperidine, 4-(1H- imidazol-4- ylmethoxy)-1-[[4- (phenylethynyl)-2- thienyl]methyl]- | 377 |
| 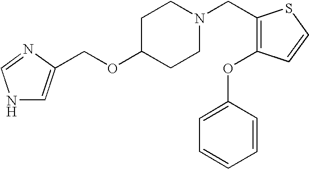 | 86 | piperidine, 4-(1H- imidazol-4- ylmethoxy)-1-[(3- phenoxy-2- thienyl)methyl]- | 369 |
| 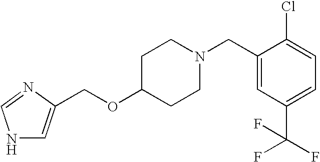 | 87 | piperidine, 1-[[2- chloro-5- (trifluoromethyl)phenyl]- methyl]-4-(1H- imidazol-4- ylmethoxy)- | 373 |
| 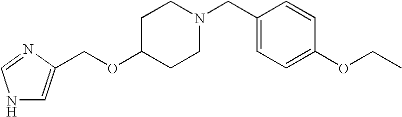 | 88 | piperidine, 4-(1H- imidazol-4- ylmethoxy)-1-[(4- propoxyphenyl)- methyl]- | 329 |
| 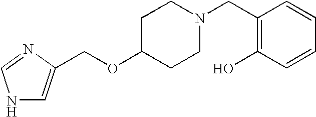 | 89 | phenol, 2-[[4-(1H- imidazol-4- ylmethoxy)-1- piperidinyl]methyl]- | 287 |
| 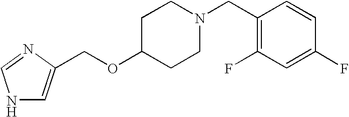 | 90 | piperidine, 1-[(2,4- difluorophenyl)methyl]- 4-(1H-imidazol-4- ylmethoxy)- | 307 |
| 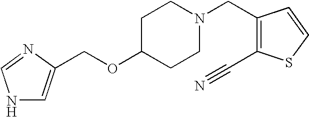 | 91 | 2- thiophenecarbonitrile, 3-[[4-(1H-imidazol-4- ylmethoxy)-1- piperidinyl]methyl]- | 302 |
| 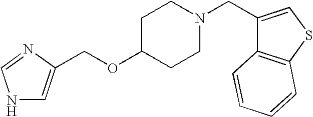 | 92 | piperidine, 1- (benzo[b]thien-3- ylmethyl)-4-(1H- imidazol-4- ylmethoxy)- | 327 |
| 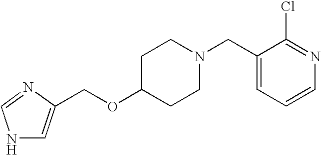 | 93 | pyridine, 2-chloro-3- [[4-(1H-imidazol-4- ylmethoxy)-1- piperidinyl]methyl]- | 306 |
| 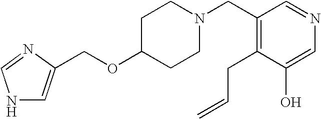 | 94 | phenol, 3-[[4-(1H- imidazol-4- ylmethoxy)-1- piperidinyl]methyl]-2- (2-propenyl)- | 327 |
| 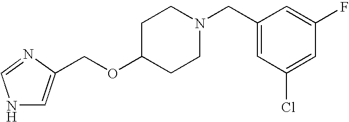 | 95 | piperidine, 1-[(4- chloro-3- fluorophenyl)methyl]- 4-(1H-imidazol-4- ylmethoxy)- | 323 |
| 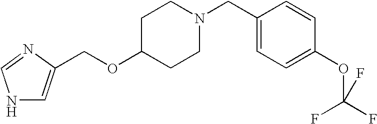 | 96 | piperidine, 4-(1H- imidazol-4- ylmethoxy)-1-[[4- (trifluoromethoxy)- phenyl]methyl]- | 355 |
| 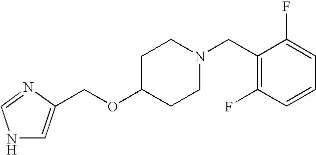 | 97 | piperidine, 1-[(2,6- difluorophenyl)methyl]- 4-(1H-imidazol-4- ylmethoxy)- | 307 |
| 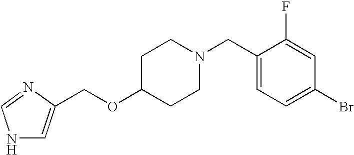 | 98 | piperidine, 1-[(4- bromo-2- fluorophenyl)methyl]- 4-(1H-imidazol-4- ylmethoxy)- | 367 |
| 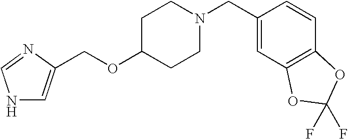 | 99 | piperidine, 1-[(2,2- difluoro-1,3- benzodioxol-5- yl)methyl]-4-(1H- imidazol-4- ylmethoxy)- | 351 |
| 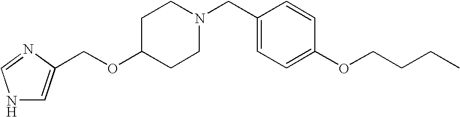 | 100 | piperidine, 1-[(4- butoxyphenyl)methyl]- 4-(1H-imidazol-4- ylmethoxy)- | 343 |
| 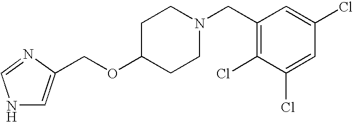 | 101 | piperidine, 4-(1H- imidazol-4- ylmethoxy)-1-[(2,3,5- trichlorophenyl)- methyl]- | 373 |
| 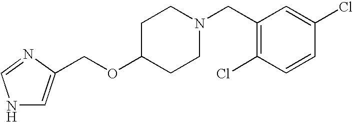 | 102 | piperidine, 1-[(2,5- dichlorophenyl)methyl]- 4-(1H-imidazol-4- ylmethoxy)- | 339 |
| 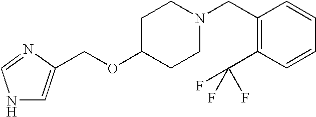 | 103 | piperidine, 4-(1H- imidazol-4- ylmethoxy)-1-[[2- (trifluoromethyl)- phenyl]methyl]- | 339 |
| 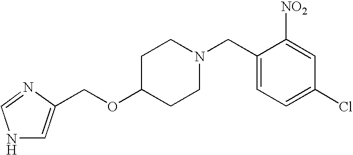 | 104 | piperidine, 1-[(4- chloro-2- nitrophenyl)methyl]- 4-(1H-imidazol-4- ylmethoxy)- | 350 |

Pharmacological Data

H4-CHO Fluorometric Imaging Plate Reader (FLIPR) Assay

FLIPR was employed to measure the intracellular calcium mobilisation to H4 receptor activation by histamine. CHO-K1 cells expressing the human recombinant H4 receptor with Gal 6 were purchased from Euroscreen and used in the experiments to identify H4 antagonists. The same protocol was used with the human H3-CHO cell line (Euroscreen) to determine selectivity of the H4 antagonists.

Briefly, the FLIPR protocol detects changes in [Ca²⁺]ᵢ using Fluo-3AM loaded cells (Schroeder & Neagle. FLIPR: A new instrument for accurate, high throughput optical screening. J. Biomol. Screening: 1(2), 75-80, 1996). The H4-CHO cells were cultured routinely in T225 cm² tissue culture flasks as monolayers in NUT Hams (with 1% (v/v) Glutamine) supplemented with 10% (v/v) heat inactivated foetal bovine serum and grown under Geneticin (1 mg/ml) antibiotic selection & 1 mg/ml Zeocin selection. Cultures were maintained at 37° C. in a humidified atmosphere of 5% CO₂ and passaged every 3 days.

H4-CHO cells were seeded at 10,000 cells/well (384 FLIPR plate) 18-24 hr before the experiment. Cells were washed to remove medium and replaced with loading buffer for 1.5 hrs. The loading buffer contains Hanks balance salt solution (Sigma), HEPES (20 mM), probenecid (2.5 mM) and Fluo 3-AM (4 μM)/Brilliant Black at pH7.4. The EC₅₀ of histamine was determined on the day of the experiment and 2×EC₅₀ was chosen as the dose to test compounds against. ATP stimulation was included in the FLIPR assay to exclude any non-selective antagonists.

Step by Step Guide to FLIPR Assay

1. Cells were harvested using 1× dissociation solution and plated onto poly-D-lysine coated FLIPR 384 plates at 1.0×10⁴ cells per well 18-24 hours prior to experiment.

2. Media was removed from the cells by tipping the plates and gently blotted onto tissue to remove any excess medium.

3. 30 μl loading buffer was added to all wells and plates were incubated for 90 min at 37° C.

4. 96 well histamine EC₅₀ plate was made and then 40 μl was indexed into 4 quadrants in a 384 well plate.

5. 96 well compound vehicle (1% DMSO) plate was made and indexed into a quadrant of a 384 well plate.

6. Plates transferred to FLIPR and run using the following 384 well protocol

7. EC₅₀ for histamine was calculated.

8. 96 well histamine plate (×10 EC₅₀) was made and then 60 μl was indexed into 4 quadrants in a 384 well plate.

9. Each 96 well compound plate was made and indexed into a quadrant of a 384 well plate.

10. ATP plate was made in a 96 well plate and then 60 μl was indexed into 4 quadrants in a 384 well plate.

11. Plates transferred to FLIPR and run using the following 384 well protocol

Cell media (but not cells) removed from FLIPR 384 plate

30 ul of loading buffer added to FLIPR 384 plate

10 μl compound added to cell plate

Reads taken for 5 min to determine compound effects

10 μl histamine added to cell plate

Reads taken to determine histamine response

10 μl ATP added to cell plate

Reads taken for 5 min to determine ATP response

12. Final assay concentrations

Compound concentration range=30 μM to 0.01 μM

histamine=2× calculated EC₅₀

ATP=11 μM

384 Well FLIPR Protocol

General

| | |
|---|---|
| Exposure | 0.4 |
| Filter | 1 |
| Presoak | None |
| Second Sequence | Yes |
| Third Sequence | YES |
| Auto prompt for notes | No |
| Auto print | Yes |
| Auto export time sequence | Yes |
| Auto export stats | Yes |

First sequence (Compound)

| | |
|---|---|
| Initial interval | 20 readings every 2 sec |
| Second interval | 27 readings every 10 sec |

Fluid Addition

| | |
|---|---|
| Addition Active | Yes |
| Volume | 10.0 |
| After Sample | 5 |
| Height | 25 |
| Speed | 15.0 |
| Mix | No |

Second sequence (histamine)

| | |
|---|---|
| Initial interval | 20 readings every 2 sec |
| Second interval | 12 readings every 10 sec |

Fluid Addition

| | |
|---|---|
| Addition Active | Yes |
| Volume | 10.0 |
| After Sample | 3 |
| Height | 35 |
| Speed | 15.0 |
| Mix | No |

Third sequence (ATP)

| | |
|---|---|
| Initial interval | 20 readings every 2 sec |
| Second interval | 12 readings every 10 sec |

Fluid Addition

| | |
|---|---|
| Addition Active | Yes |
| Volume | 10.0 |
| After Sample | 3 |
| Height | 35 |
| Speed | 40.0 |
| Mix | No |

Pipetting

| | |
|---|---|
| Mix Volume | 0.0 |
| Mix Cycles | 0 |
| Leave tips in well | No |
| Remove fluid after addition | No |

Stage heated to 35° C.

The compounds of the examples have an IC₅₀ values vs H4 of <10 micromolar.

1-8. (canceled)

9. A compound of formula (IA): 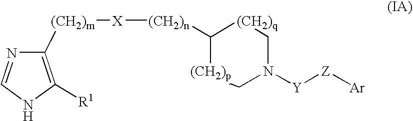
in which:
Ar is an aryl group, a 5-7 membered heteraromatic ring containing 1-4 heteroatoms selected from nitrogen, oxygen or sulphur, or a bicyclic or tricyclic heteraromatic ring containing 1-4 heteroatoms selected from nitrogen, oxygen or sulphur, each of which aryl group or heteroaromatic ring can be optionally substituted by 1-3 groups selected from C₁₋₆ alkyl, C₁₋₆ alkylthio, C₁₋₆ alkoxy, halogen, cyano, CF₃, OCF₃, C₃₋₆ cycloalkyl, C₂₋₆ alkenyl, C₂₋₆ alkynyl, C₂₋₆-alkenyloxy, hydroxyl, nitro, tosyl, thienyl, benzyl, phenyl, nitrophenyl,
R¹ is hydrogen or C₁₋₆alkyl;
X is O, NR², CH₂ or SOₓ
R² is C₁₋₆ alkyl;
x is 0, 1 or 2;
Y is C═O, SO₂, or (C═O)NH;
Z is (CR³R⁴)ᵣ or Y and Z together form a CH═CH group;
m and n are independently 0, 1, 2 or 3;
p and q are independently 0, 1 or 2;
r is 0, 1, 2, 3, or 4, and
R³ and R⁴ are independently hydrogen or C₁₋₆alkyl.

10. A compound according to claim 9 in which Y is C═O.

11. A pharmaceutical composition comprising a compound of formula (IA) according to claim 9, or a pharmaceutically acceptable salt or solvate thereof, in association with a pharmaceutically acceptable adjuvant, diluent or carrier.

12-15. (canceled)

16. A method for the treatment of diseases mediated by histamine H3 and H4 receptors
comprising administering to a subject in need thereof a therapeutically effective amount of a compound of formula (I) or a pharmaceutically acceptable salts or a solvate thereof: 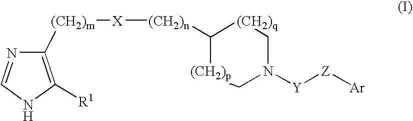
in which:
Ar is an aryl group, a 5-7 membered heteraromatic ring containing 1-4 heteroatoms selected from nitrogen, oxygen or sulphur, or a bicyclic or tricyclic heteraromatic ring containing 1-4 heteroatoms selected from nitrogen, oxygen or sulphur, each of which aryl group or heteroaromatic ring can be optionally substituted by 1-3 groups selected from C₁₋₆ alkyl, C₁₋₆ alkylthio, C₁₋₆ alkoxy, halogen, cyano, CF₃, OCF₃, C₃₋₆ cyclolalkyl, C₂₋₆ alkenyl, C₁₋₆ alkynyl, C₂₋₆ alkenyloxy, hydroxyl, nitro, tosyl, thienyl, benzyl, phenyl, nitrophenyl,
R¹ is hydrogen or C₁₋₆ alkyl;
X is O, NR², CH₂ or SOₓ
R² is C₁₋₆ alkyl;
x is 0, 1 or 2;
Y is CH₂, C═O, SO₂, or (C═O)NH;
Z is (CR³R⁴)ᵣ or Y and Z together form a CH═CH group;
m and n are independently 0, 1, 2 or 3;
p and q are independently 0, 1 or 2;
r is 0, 1, 2, 3, or 4 and
R³ and R⁴ are independently hydrogen or C₁₋₆alkyl.

17. The method according to claim 16 wherein in said compound:
Ar¹ is phenyl optionally substituted with iodo, chloro and fluoro, cyclohexyl, methyl, ethyl, propyl, t-butyl, ethynyl, propenyloxyl, hydroxyl, methoxyl, nitro, tosyl, trifluoromethyl, thienyl, benzyl, cyano, phenylethynyl, nitrophenyl, methylthio, propoxyl, butoxyl, 2-propenyl, or trifluormethoxyl.

18. The method according to claim 16 wherein in said compound:
R¹ is hydrogen or methyl.

19. The method according to claim 16 wherein in said compound:
X is O.

20. The method according to claim 16 wherein in said compound:
Y is CH₂ or C═O and Z is CH₂, CHMe, CH₂CHMe; or Y and Z form a CH═CH group.

21. The method according to claim 16 wherein in said compound:
m is 1 and n is 0.

22. The method according to claim 16 wherein in said compound:
p and q are both 1.

23. The method according to claim 16 wherein said compound is selected from:
4-(1H-Imidizol-4-ylmethoxy)-1-(1-oxo-3-phenylbutyl)-piperidine;
4-(1H-Imidazol-4-ylmethoxy)-1-[[4-(trifluoromethyl)phenyl]acetyl]-piperidine;
1-[2-(4-Hydroxyphenyl)-1-oxopropyl]-4-[(5-methyl-1H-imidazol-4-yl)methoxy]-piperidine;
1-[(4-fluorophenyl)acetyl]-4-(1H-imidazol-4-ylmethoxy)-piperidine;
1-[(2-chlorophenyl)acetyl]-4-(1H-imidazol-4-ylmethoxy)-piperidine;
1-[(4-chlorophenyl)acetyl]-4-(1H-imidazol-4-ylmethoxy)-piperidine;
4-(1H-imidazol-4-ylmethoxy)-1-(phenylacetyl)-piperidine;
1-(4-cyclohexylbenzoyl)-4-(1-imidazol-4-ylmethoxy)-piperidine;
1-[(3,4-dichlorophenyl)acetyl]-4-(1H-imidazol-4-ylmethoxy)-piperidine;
4-(1H-imidazol-4-ylmethoxy)-1-[(4-methylphenyl)acetyl]-piperidine;
1-[(3,4-difluorophenyl)acetyl]-4-(1H-imidazol-4-ylmethoxy)-piperidine;
1-[(2,4-difluorophenyl)acetyl]-4-(1H-imidazol-4-ylmethoxy)-piperidine
4-(1H-imidazol-4-ylmethoxy)-1-[(4′-propyl[1,1′-biphenyl]-4-yl)carbonyl]-piperidine;
1-[2-(4-hydroxyphenyl)-1-oxopropyl]-4-(1H-imidazol-4-ylmethoxy)-piperidine;
1-[(2E)-3-(3,4-dichlorophenyl)-1-oxo-2-propenyl]-4-(1H-imidazol-4-ylmethoxy)-piperidine;
1-[3-(2,4-dichlorophenyl)-1-oxopropyl]-4-(1H-imidazol-4-ylmethoxy)-piperidine;
1-[(2,4-dichlorophenyl)acetyl]-4-(1H-imidazol-4-ylmethoxy)-piperidine;
1-[(2-Bromophenyl)methyl]-4-(1H-imidazol-4-ylmethoxy)-piperidine;
1-[(3-Bromo-2-thienyl)methyl]-4-[(5-methyl-1H-imidazol-4-yl)methoxy]-piperidine;
1-[(3-bromo-2-thienyl)methyl]-4-(1H-imidazol-4-ylmethoxy)piperidine;
1-[(4-ethynylphenyl)methyl]-4-(1H-imidazol-4-ylmethoxy)-piperidine;
4-(1H-imidazol-4-ylmethoxy)-1-[[3-(4-methylphenoxy)phenyl]methyl]-piperidine;
4-(1H-imidazol-4-ylmethoxy)-1-[4[(2-propenyloxy)phenyl]methyl]-piperidine;
4-[[4-(1H-imidazol-4-ylmethoxy)-1-pipendinyl]methyl]-phenol;
4-(1H-imidazol-4-ylmethoxy)-1-[(2-methoxyphenyl)methyl]-piperidine;
4-(1H-imidazol-4-ylmethoxy)-1-[[3-(4-methoxyphenoxy)phenyl]methyl]-piperidine;
1-[(2,3-dichlorophenyl)methyl]-4-(1H-imidazol-4-ylmethoxy)-piperidine;
1-[(2-chloro-4-fluorophenyl)methyl]-4-(1H-imidazol-4-ylmethoxy)-piperidine;
1-(2-dibenzofuranylmethyl)-4-(1H-imidazol-4-ylmethoxy)-piperidine;
4-(1H-imidazol-4-ylmethoxy)-1-[[2-(methylthio)phenyl]methyl]-piperidine;
4-(1H-imidazol-4-ylmethoxy)-1-(thieno[2,3-b][1]benzothien-2-ylmethyl)-piperidine;
1-[(2-chloro-5-nitrophenyl)methyl]-4-(1H-imidazol-4-ylmethoxy)piperidine;
1H-pyrrole, 2-[[4-(1H-imidazol-4-ylmethoxy)-1-piperidinyl]methyl]-1-[(4-methylphenyl)sulfonyl]-;
2-ethoxy-6-[[4-(1H-imidazol-4-ylmethoxy)-1-piperidinyl]methyl]-phenol;
1-(1,3-benzodioxol-5-ylmethyl)-4-(1H-imidazol-4-ylmethoxy)-piperidine;
4-(1H-imidazol-4-ylmethoxy)-1-[[4-(phenylmethoxy)phenyl]methyl]-piperidine;
1-[[2-fluoro-4-(trifluoromethyl)phenyl]methyl]-4-(1H-imidazol-4-ylmethoxy)-piperidine;
1-[(4-bromophenyl)methyl]-4-(1H-imidazol-4-ylmethoxy)-piperidine;
4-(1H-imidazol-4-ylmethoxy)-1-[(4-methylphenyl)methyl]-piperidine;
4-(1H-imidazol-4-ylmethoxy)-1-(2-thienylmethyl)-piperidine;
1-[(4-chlorophenyl)methyl]-4-(1H-imidazol-4-ylmethoxy)-piperidine;
1-[(2-chloro-6-fluorophenyl)methyl]-4-(1H-imidazol-4-ylmethoxy)-piperidine;
4-(1H-imidazol-4-ylmethoxy)-1-[(3-methyl-2-thienyl)methyl]-piperidine;
4-(1H-imidazol-4-ylmethoxy)-1-(2-naphthalenylmethyl)-piperidine;
4-(1H-imidazol-4-ylmethoxy)-1-(1-naphthalenylmethyl)-piperidine;
4-(1H-imidazol-4-ylmethoxy)-1-[(2-nitrophenyl)methyl]-piperidine;
4-(1H-imidazol-4-ylmethoxy)-1-(3-thienylmethyl)-piperidine;
1-([1,1′-biphenyl]-4-ylmethyl)-4-(1H-imidazol-4-ylmethoxy)-piperidine;
1-[(2,5-difluorophenyl)methyl]-4-(1H-imidazol-4-ylmethoxy)-piperidine;
4-(1H-imidazol-4-ylmethoxy)-1-[(3-phenoxyphenyl)methyl]-piperidine;
4-(1H-imidazol-4-ylmethoxy)-1-[(3-methylphenyl)methyl]-piperidine;
1-(2-furanylmethyl)-4-(1H-imidazol-4-ylmethoxy)-piperidine;
1-[(2,6-dichlorophenyl)methyl]-4-(1H-imidazol-4-ylmethoxy)-piperidine;
1-[(4-fluorophenyl)methyl]-4-(1H-imidazol-4-ylmethoxy)-piperidine;
1-[(3-fluorophenyl)methyl]-4-(1H-imidazol-4-ylmethoxy)-piperidine;
1-(3-furanylmethyl)-4-(1H-imidazol-4-ylmethoxy)-piperidine;
1-[(4-ethylphenyl)methyl]-4-(1H-imidazol-4-ylmethoxy)-piperidine;
4-(1H-imidazol-4-ylmethoxy)-1-[(2-methylphenyl)methyl]-piperidine;
1-[(3-chlorophenyl)methyl]-4-(1H-imidazol-4-ylmethoxy)-piperidine;
4-(1H-imidazol-4-ylmethoxy)-1-[(5-methyl-2-thienyl)methyl]-piperidine;
1-[(4-bromo-2-thienyl)methyl]-4-(1H-imidazol-4-ylmethoxy)-piperidine;
1-([2,2′-bithiophen]-5-ylmethyl)-4-(1H-imidazol-4-ylmethoxy)-piperidine;
3,5-dichloro-2-[[4-(1H-imidazol-4-ylmethoxy)-1-piperidinyl]methyl]-phenol;
1-[(3,4-difluorophenyl)methyl]-4-(1H-imidazol-4-ylmethoxy)-piperidine;
1-[(3,5-difluorophenyl)methyl]-4-(1H-imidazol-4-ylmethoxy)-piperidine;
1-[(6-chloro-1,3-benzodioxol-5-yl)methyl]-4-(1H-imidazol-4-ylmethoxy)-piperidine;
1-[[4-[4-(1,1-dimethylethyl)-2-thiazolyl]phenyl]methyl]-4-(1H-imidazol-4-ylmethoxy)-piperidine;
4-(1H-imidazol-4-ylmethoxy)-1-[(1-methyl-1H-pyrrol-2-yl)methyl]-piperidine;
1H-indole, 3-[[4-(1H-imidazol-4-ylmethoxy)-1-piperidinyl]methyl]-1-(phenylmethyl);
1-[(5-chloro-2-thienyl)methyl]-4-(1H-imidazol-4-ylmethoxy)-piperidine;
1-(1,3-benzodioxol-4-ylmethyl)-4-(1H-imidazol-4-ylmethoxy)-piperidine;
2-thiophenecarbonitrile, 3-[[4-[[4-(1H-imidazol-4-ylmethoxy)-1-piperidinyl]methyl]phenoxy]methyl]-piperidine;
4-(1H-imidazol-4-ylmethoxy)-1-[[5-(phenylethynyl)-2-thienyl]methyl]-piperidine;
4-(1H-imidazol-4-ylmethoxy)-1-[[5-(4-nitrophenyl)-2-furanyl]methyl]-piperidine;
4-(1H-imidazol-4-ylmethoxy)-1-[[5-(3-nitrophenyl)-2-furanyl]methyl]-piperidine;
1-[(4-chloro-1H-pyrazol-3-yl)methyl]-4-(1H-imidazol-4-ylmethoxy)-piperidine;
1-[(4-bromo-1-methyl-1H-pyrazol-3-yl)methyl]-4-(1H-imidazol-4-ylmethoxy)-piperidine;
1-[(4-bromo-1H-pyrazol-3-yl)methyl]-4-(H-imidazol-4-ylmethoxy)-piperidine;
2-[[4-(1H-imidazol-4-ylmethoxy)-1-piperidinyl]methyl]-benzonitrile;
4-(1H-imidazol-4-ylmethoxy)-1-[(4-iodophenyl)methyl]-piperidine;
1-[(5-ethyl-2-thienyl)methyl]-4-(1H-imidazol-4-ylmethoxy)-piperidine;
4-(1H-imidazol-4-ylmethoxy)-1-[[5-(methylthio)-2-thienyl]methyl]-piperidine;
1-[[1-(3,5-dichlorophenyl)-1H-pyrrol-2-yl]methyl]-4-(1H-imidazol-4-ylmethoxy)-piperidine;
1-[[1-(4-chlorophenyl)-1H-pyrrol-2-yl]methyl]-4-(1H-imidazol-4-ylmethoxy)-piperidine;
4-(1H-imidazol-4-ylmethoxy)-1-[[4-(phenylethynyl)-2-thienyl]methyl]-piperidine;
4-(1H-imidazol-4-ylmethoxy)-1-[(3-phenoxy-2-thienyl)methyl]-piperidine;
1-[[2-chloro-5-(trifluoromethyl)phenyl]methyl]-4-(1H-imidazol-4-ylmethoxy)-piperidine;
4-(1H-imidazol-4-yl methoxy)-1-[(4-propoxyphenyl)methyl]-piperidine;
2-[[4-(1H-imidazol-4-ylmethoxy)-1-piperidinyl]methyl]-phenol;
1-[(2,4-difluorophenyl)methyl]-4-(1H-imidazol-4-ylmethoxy)-piperidine;
3-[[4-(1H-imidazol-4-ylmethoxy)-1-piperidinyl]methyl]-2-thiophenecarbonitrile;
1-(benzo[b]thien-3-ylmethyl)-4-(1H-imidazol-4-ylmethoxy)-piperidine;
2-chloro-3-[[4-(1H-imidazol-4-ylmethoxy)-1-piperidinyl]methyl]-pyridine;
3-[[4-(1H-imidazol-4-ylmethoxy)-1-piperidinyl]methyl]-2-(2-propenyl)-phenol;
1-[(4-chloro-3-fluorophenyl)methyl]-4-(1H-imidazol-4-ylmethoxy)-piperidine;
4-(1H-imidazol-4-ylmethoxy)-1-[[4-(trifluoromethoxy)phenyl]methyl]-piperidine;
1-[(2,6-difluorophenyl)methyl]-4-(1H-imidazol-4-ylmethoxy)-piperidine;
1-[(4-bromo-2-fluorophenyl)methyl]-4-(1H-imidazol-4-ylmethoxy)-piperidine;
1-[(2,2-difluoro-1,3-benzodioxol-5-yl)methyl]-4-(1H-imidazol-4-ylmethoxy)-piperidine;
1-[(4-butoxyphenyl)methyl]-4-(1H-imidazol-4-ylmethoxy)-piperidine;
4-(1H-imidazol-4-ylmethoxy)-1-[(2,3,5-trichlorophenyl)methyl]-piperidine;
1-[(2,5-dichlorophenyl)methyl]-4-(1H-imidazol-4-ylmethoxy)-piperidine;
4-(1H-imidazol-4-ylmethoxy)-1-[[2-(trifluoromethyl)phenyl]methyl]-piperidine, or
1-[(4-chloro-2-nitrophenyl)methyl]-4-(1H-imidazol-4-ylmethoxy)-piperidine
or a pharmaceutically acceptable salt or solvate thereof.
